(19) United States
(12) Patent Application Publication
(10) Pub. No.: US 2024/0024651 A1
(43) Pub. Date: Jan. 25, 2024

(54) ACCESS PORT CAP SYSTEM AND METHOD OF USE

(71) Applicants: Arlene Joyce Rosetta-Rangel, Littleton, CO (US); Joel Hughes, Erie, CO (US)

(72) Inventors: Arlene Joyce Rosetta-Rangel, Littleton, CO (US); Joel Hughes, Erie, CO (US)

(21) Appl. No.: 18/213,060

(22) Filed: Jun. 22, 2023

Related U.S. Application Data
(60) Provisional application No. 63/390,879, filed on Jul. 20, 2022.

(51) Int. Cl. A61M 39/02

(52) U.S. Cl. CPC A61M 39/0247; A61M 2039/0288; A61M 2039/027; A61M 2039/0261

(57) ABSTRACT

An access port cap system includes a cap having a body forming an interior reservoir therein, and a perimeter lip extending from a base of the body; and an adhesive member having a body forming an opening therein, and a double-sided adhesive tape contained between a top release liner and a bottom release liner; wherein the body of the adhesive member is configured to surround the circumference of an implanted port.

### CROSS REFERENCE TO RELATED APPLICATIONS

This application claims the benefit of U.S. Provisional Application 63/390,879, filed Jul. 20, 2022, of which is hereby incorporated by reference in its entirety.

### BACKGROUND

### 1. Field of the Invention

The present invention relates generally to topical application of numbing cream systems, and more specifically, to an access port cap for application of a numbing cream topically.

### 2. Description of Related Art

In medical systems, an access port is an implanted device which allows easy access to a patient's veins. The access port is surgically inserted completely beneath the skin and consists of a portal and a catheter. The portal is typically made from a silicone bubble and appears as a small bump under the skin, wherein the silicone allows for the portal to be punctured repeatedly without compromise.

Prior to port access, it is common to use a numbing agent or cream to improve patient comfort. Conventional methods utilize a numbing cream that is placed over the port and covered such as with cellophane and tape. This method is limited in effectiveness as the numbing agent is applied in only a thin layer, which does not penetrate to numb the puncture area and leads to patient pain when port access occurs. In addition, conventional methods do not prevent the numbing agent or cream from smearing or being dispersed to places other than over the port access area.

Accordingly, it is an object of the present invention to provide for an access port cap system and method that allows for the application of a thick layer of a numbing agent, thereby providing a means to improve patient comfort during port access. It will be understood and appreciated that the system and method of the present invention is not limited to port access and may be applied to alternative uses, wherein topical numbing is desired.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a top view of a single access port cap system in accordance with one or more embodiments of the present invention;

FIG. 2 is an exploded view of the single access port cap system of FIG. 1;

FIG. 3 is an exploded view of the adhesive member of FIG. 2;

FIG. 4 is a cross-sectional view of the single access port cap system of FIG. 1 in use;

FIG. 5 is a top view of a double access port cap system in accordance with one or more embodiments of the present invention;

FIG. 6 is an exploded view of the double access port cap system of FIG. 5;

FIG. 7 is an exploded view of the adhesive member of FIG. 6; and

FIG. 8 is a flowchart of a method of use of a single access port cap system in accordance with one or more embodiments of the present invention.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional systems and methods of topical application of a numbing agent. Specifically, the present invention utilizes a cap, wherein the cap encapsulates a thick layer of numbing cream to improve the effectiveness of numbing the area and improving patient comfort during port access. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

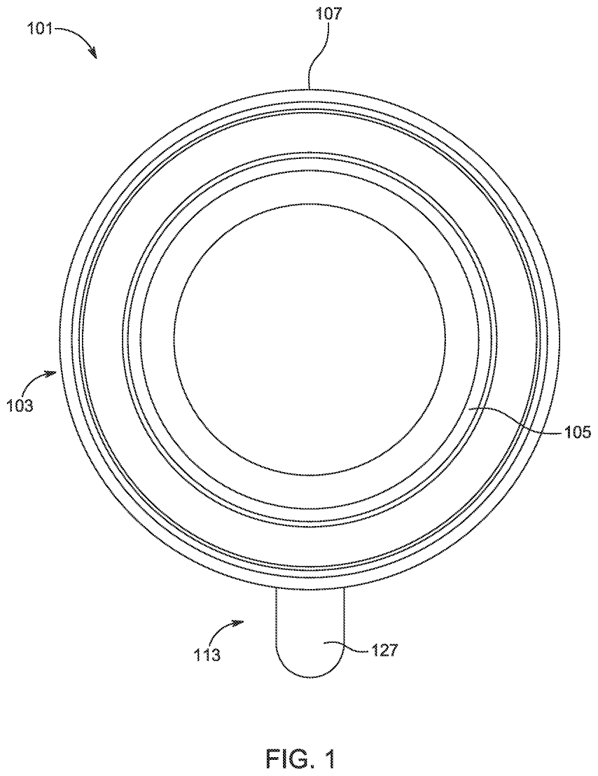

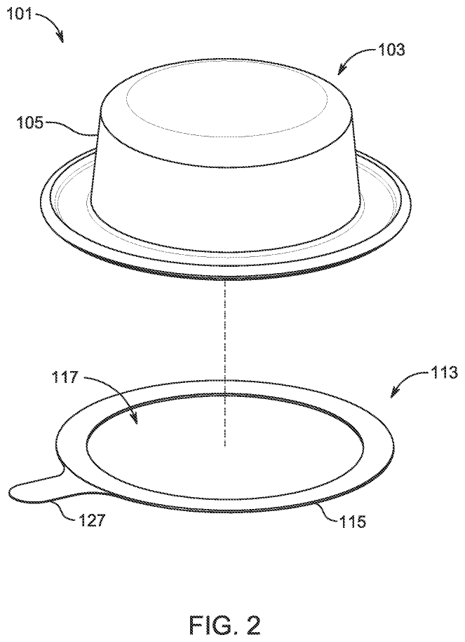

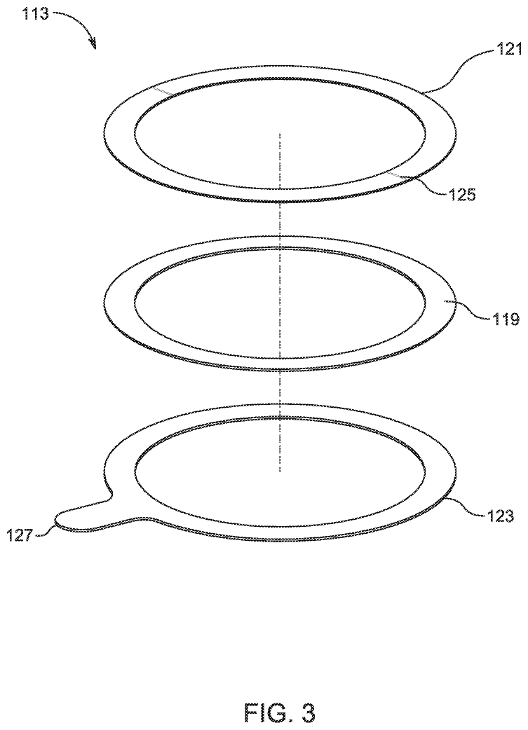

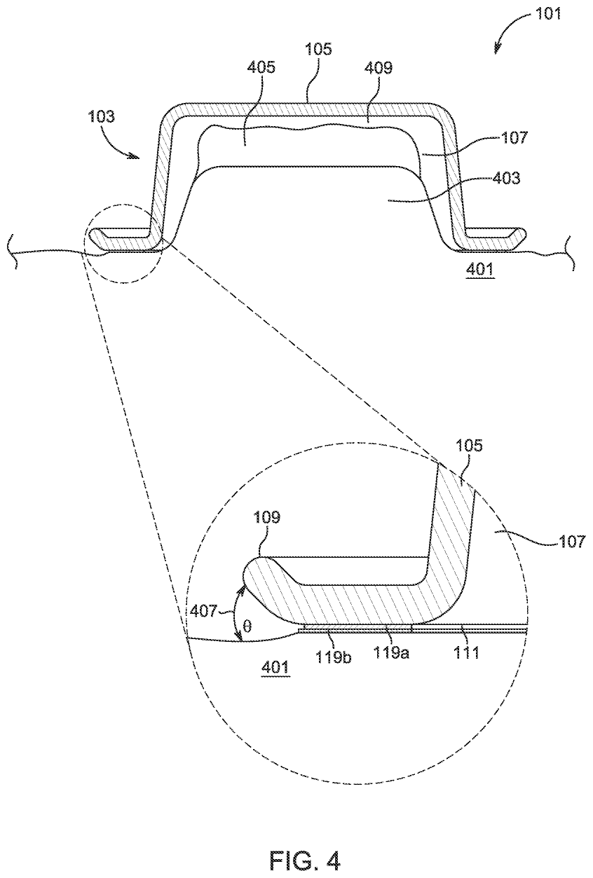

Referring now to the drawings wherein like reference characters identify corresponding or similar elements throughout the several views, FIGS. 1 through 4 depict various views of a single access port cap system 101 in accordance with one or more embodiments of the present application. It will be appreciated that the system of the present invention overcomes one or more of the above-listed problems commonly associated with conventional systems.

In the contemplated embodiment, the single access port cap system 101 includes a cap 103 having a dome-shaped body 105 forming an interior reservoir 107 therein. The cap 103 also includes a perimeter lip 109 that extends from a base 111 of the body 105. The lip 109 extends at an angle 407, (e.g., preferably a 45-degree angle) to provide for ease of removal after use of the cap 103. It should be noted that the cap 103 enables the single access port cap system 101 to be reusable, wherein the user may wash and reuse the cap 103 multiple times.

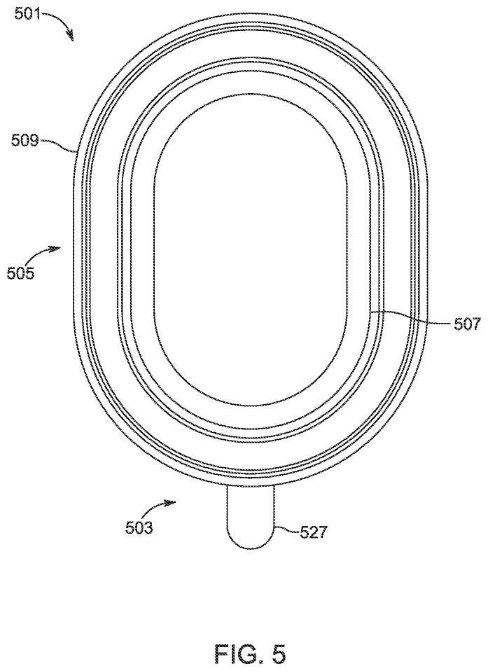

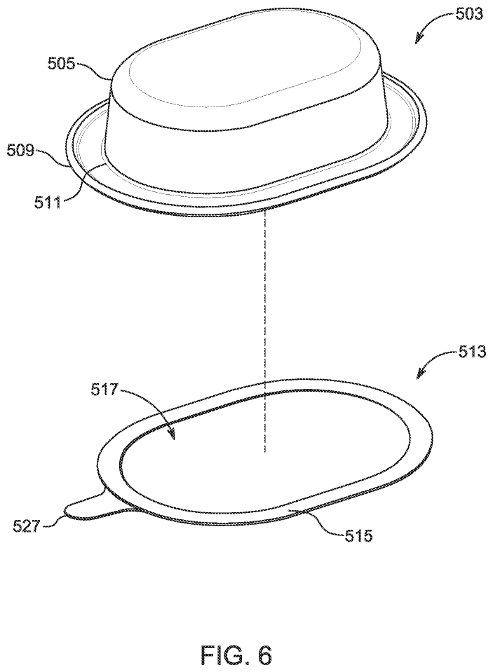

It should be appreciated that the shape of the body 105 can vary in size and shape, including a rectangular, triangular, circular, oval (as shown in FIGS. 5 and 6), or other suitably shaped configuration.

It should be appreciated that in some embodiments, the single access port cap system 101 may include ventilation features such as holes, slots, or the like. In other embodiments, the single access port cap system 101 may eliminate ventilation features such that the configuration may prevent water or other liquids from breaching therein.

The single access port cap system 101 also includes an adhesive member 113 having a body 115 forming an opening 117 therein. The adhesive member 113 comprises of a double-sided adhesive tape 119 contained between a top release liner 121 and a bottom release liner 123. One contemplated material for the adhesive tape 119 is disposable medical grade double sticky tape.

In some embodiments associated with manufacturing purposes, the top release liner 121 can include scored edging 125 for ease of assembly. It should be noted that the adhesive member 113 in such embodiments enables the single access port cap system 101 to be disposable.

In other embodiments associated with consumer use, the adhesive member 113 enables the single access port cap system 101 to be reusable. For example, the adhesive member 113 can include the top release liner 121 and the scored edging 125, and a tab 127 coupled to the bottom release liner 123 for easy removal by the consumer. After the consumer uses the single access port cap system 101, the adhesive member 113 is removed, discarded, and replaced with a new adhesive member, and the new adhesive member is attached to the single access port cap system 101 to be used again by the consumer.

It should be appreciated that in some embodiments, the adhesive member 113 may be eliminated, and an adhesive is present on the lip 109, such that the adhesive secures the cap 103 in place during use.

During manufacturing purposes and/or consumer use, the top release liner 121 is removed, exposing the top sticky layer 119a of the double-sided adhesive tape 119. The perimeter lip 109 adheres to the exposed sticky layer 119a, thereby coupling the cap to the adhesive member 113. During consumer use, a relatively thick layer of numbing agent 405 can be placed over a raised portion of an implanted port 403 or other desired location. It should be understood that the numbing agent 405 can be contained within a variety of vehicles such as a cream, a gel, an ointment, or the like. Then, the bottom release liner 123 is removed, exposing the bottom sticky layer 119b of the double-sided adhesive tape 119. The exposed sticky layer 119b is positioned to surround the circumference of the implanted port 403 or other desired location, thereby placing the cap 103 over the implanted port 403 and by extension, the numbing agent 405. It should be noted that interior reservoir 107 is configured to provide clearance 409 between the body 105 and the numbing agent 405. The cap 103 will remain for a predetermined period of time, such as one hour or as recommended by a treating physician. During that time, the numbing agent 405 will absorb topically into the user's skin 401, thereby providing for improved numbing and improved comfort during port access. The access port cap system 101 can then be removed to allow the implanted port 403 to be accessed with a needle.

It should be appreciated that the cap 103 prevents the numbing agent 405 from spreading and coming into contact with a patient's clothing, which can cause damage to the clothing. In addition, it should be appreciated that the cap 103 holds the thick layer of numbing agent 405 over the raised portion of the implanted port 403 or desired location, thereby allowing for full penetration and maximum numbing of the area. The cap 103 provides for additional benefits, including saving time, reducing mess, infection protection, and is easy to use and apply.

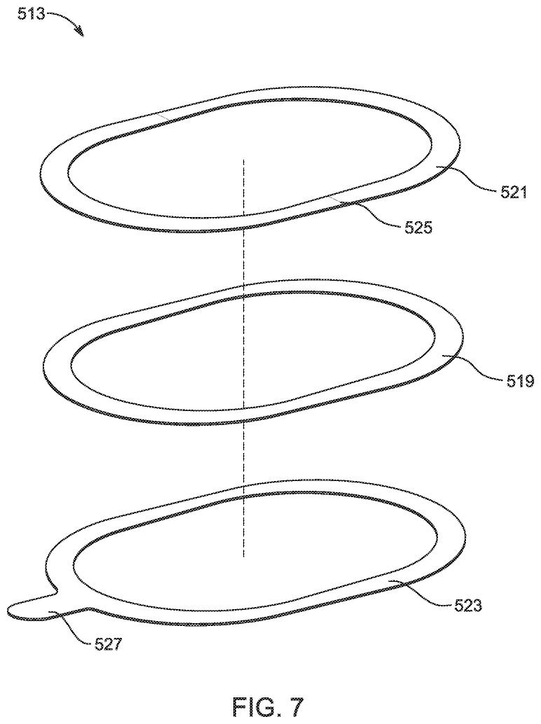

In FIGS. 5 through 7, various views of a double access port cap system 501 are illustrated. It should be understood that the access port cap system 501 is substantially similar to the access port cap system 101. The access port cap system 501 comprises of a cap 503 having a generally oval-shaped body 505 forming an interior reservoir 507 therein. The cap 503 also includes a perimeter lip 509 that extends from a base 511 of the body 505. In addition, the access port cap system 501 includes an adhesive member 513 having a body 515 forming an opening 517 therein. Like the adhesive member 113, the adhesive member 513 comprises of a double-sided adhesive tape 519 contained between a top release liner 521 and a bottom release liner 523. In addition, the top release liner 521 can include scored edging 527 for easy removal, and the bottom release liner 523 can include a tab 525 for easy removal.

Although the exemplary embodiment discussed herein is directed to port access, it should be appreciated that the present invention may be used for various other purposes, such as diabetes injections, tattoos, steroid injections, intravenous injections, intramuscular injections, external skin biopsies, hormone injections, methadone injections, infection protection, or any other suitable use.

It should also be appreciated that one of the unique features believed characteristic of the present application is that the cap provides for enough clearance to allow the application of a thick amount of the numbing agent above an implanted port, such that the thick layer can penetrate the skin over a period of time. Again, this feature provides for improved patient comfort and quality of life during port access.

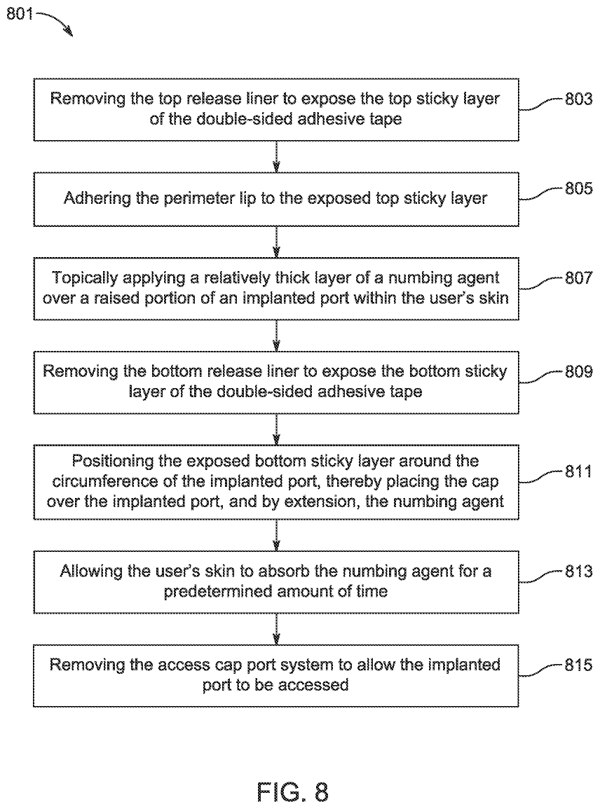

In FIG. 8, a flowchart 801 depicts a method of use of a single access port cap system, such as system 101. First, the top release liner is removed to expose the top sticky layer of the double-sided adhesive tape, step 803. The perimeter lip is then adhered to the exposed top sticky layer, step 805. It should be noted that the aforementioned steps 803, 805 describe a reusable single access port cap system.

A relatively thick layer of a numbing agent is topically applied over a raised portion of an implanted port within the user's skin, step 807. The bottom release liner is then be removed to expose the bottom sticky layer of the double-sided adhesive tape, step 809. The exposed bottom sticky layer is positioned around the circumference of the implanted port, thereby placing the cap over the implanted port, and by extension, the numbing agent, step 811. The user's skin is allowed to absorb the numbing agent for a predetermined amount of time, step 813. The access cap port system can then be removed to allow the implanted port to be accessed, step 815. It should be noted that the aforementioned steps 807-815 describe a disposable single access port cap system.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. An access port cap system, comprising:
a cap, the cap having:
a body forming an interior reservoir therein; and
a perimeter lip extending from a base of the body;
wherein the interior reservoir is configured to provide clearance between the body and a top portion of an implanted port;
wherein the perimeter lip extends at an angle for ease of removal; and
an adhesive member, the adhesive member having:
a body forming an opening therein, the body configured to surround the circumference of an implanted port; and
a double-sided adhesive tape having a top sticky layer and a bottom sticky layer, the double-sided adhesive tape contained between a top release liner and a bottom release liner;
wherein, upon removal of the top release liner, the perimeter lip adheres to the top sticky layer of the double-sided adhesive tape;
wherein, upon removal of the bottom release liner, the bottom sticky layer of the double-sided adhesive tape is positioned around the circumference of the implanted port, thereby placing the cap over the implanted port.

2. The system of claim 1, wherein the cap's body is dome-shaped or oval-shaped.

3. The system of claim 1, wherein the perimeter lip extends at an angle measured approximately 45-degrees.

4. The system of claim 1, wherein the double-sided adhesive tape is composed of medical grade double sticky tape.

5. An access port cap system, comprising:
a cap, the cap having:
a body forming an interior reservoir therein; and
a perimeter lip extending from a base of the body;
wherein the interior reservoir is configured to provide clearance between the body and a top portion of an implanted port;
wherein the perimeter lip extends at an angle measured approximately 45-degrees for ease of removal; and
an adhesive member, the adhesive member having:
a body forming an opening therein, the body configured to surround the circumference of an implanted port; and
a double-sided adhesive tape having a top sticky layer and a bottom sticky layer, the double-sided adhesive tape contained between a top release liner and a bottom release liner;
wherein the double-sided adhesive tape is composed of medical grade double sticky tape;
wherein, upon removal of the top release liner, the perimeter lip adheres to the top sticky layer of the double-sided adhesive tape;
wherein, upon removal of the bottom release liner, the bottom sticky layer of the double-sided adhesive tape is positioned around the circumference of the implanted port, thereby placing the cap over the implanted port.

6. The system of claim 5, wherein the cap's body is dome-shaped or oval-shaped.

7. A method for preventing pain when an implanted port is accessed, the method comprising:
providing an access port cap system, the access port cap system comprising:
a cap, the cap having:
a body forming an interior reservoir therein; and
a perimeter lip extending from a base of the body;
wherein the interior reservoir is configured to provide clearance between the body and a top portion of an implanted port;
wherein the perimeter lip extends at an angle for ease of removal; and
an adhesive member, the adhesive member having:
a body forming an opening therein, the body configured to surround the circumference of an implanted port; and
a double-sided adhesive tape having a top sticky layer and a bottom sticky layer, the double-sided adhesive tape contained between a top release liner and a bottom release liner;
wherein, upon removal of the top release liner, the perimeter lip adheres to the top sticky layer of the double-sided adhesive tape;
wherein, upon removal of the bottom release liner, the bottom sticky layer of the double-sided adhesive tape is positioned around the circumference of the implanted port, thereby placing the cap over the implanted port;
removing the top release liner to expose the top sticky layer of the double-sided adhesive tape;
adhering the perimeter lip to the exposed top sticky layer;
topically applying a relatively thick layer of a numbing agent over a raised portion of an implanted port within the user's skin;
removing the bottom release liner to expose the bottom sticky layer of the double-sided adhesive tape;
positioning the exposed bottom sticky layer around the circumference of the implanted port, thereby placing the cap over the implanted port, and by extension, the numbing agent;
allowing the user's skin to absorb the numbing agent for a predetermined amount of time; and
removing the access cap port system to allow the implanted port to be accessed.
